(19) United States
(12) Patent Application Publication
(10) Pub. No.: US 2011/0104238 A1
(43) Pub. Date: May 5, 2011

(54) WETTABLE FILLERS FOR IMPROVED RELEASE OF HYDROPHILIC MATERIALS FROM CHEWING GUM COMPOSITIONS

(76) Inventors: Michael S. Haas, Naperville, IL (US); Darei C. Biesczat, Crown Point, IN (US); Pamela M. Mazurek, Orland Park, IL (US)

(21) Appl. No.: 12/867,178

(22) PCT Filed: Feb. 10, 2009
(86) PCT No.: PCT/US2009/033643

§ 371 Date: Dec. 7, 2010

Related U.S. Application Data
(60) Provisional application No. 61/029,227, filed on Feb. 15, 2008.

(51) Int. Cl. A61K 9/68; A61K 33/42; A61P 19/00; A23G 4/00; A23G 4/06; A23G 4/20

(52) U.S. Cl. 424/440; 424/48; 424/602; 426/3; 426/5

(57) ABSTRACT

A chewing gum is provided in the present disclosure. In an embodiment, the chewing gum comprises at least one wettable filler wherein the wettable filler aids in producing an increasing release of one or more hydrophilic additives. In another embodiment, the chewing gum comprises a wettable filler having a γ⁻ in a range of at least 15.0 mJ/m² to about 65.0 mJ/m², wherein the wettable filler aids in increasing the release of at least one hydrophilic additive. A method for increasing the release of one or more hydrophilic additives in a chewing gum composition is also provided.

### BACKGROUND

The present disclosure generally relates to chewing gum compositions. More specifically, the present disclosure relates to chewing gum compositions that use fillers to promote optimal release of substances having hydrophilic properties.

Most chewing gums including an additive and/or medicament having hydrophilic properties often experience incomplete release and/or retardation of late term release. This is due to the additive(s) binding to the chewing gum base portion due to the base's hydrophobic nature. Further, the ineffective release of these additives may stem from the additive continuously reabsorbing into the chewing gum matrix during mastication. As a result, the hydrophilic additive never completely releases from the chewing gum composition, thus offering less then optimal sensorial characteristics and/or health benefits.

For example, the problem of incomplete release and/or retardation of late term release occurs when applying salts to chewing gum for the benefit of tooth remineralization. Chewing gums containing salts, such as calcium and phosphate salts, for tooth remineralization experience both an incomplete release and retardation of late term release, thus never completely releasing from the chewing gum composition offering less than optimal remineralization benefits to the consumer.

Generally, to compensate for this reduced release of partially to fully hydrophilic materials from chewing gums, developers formulate alternative means to promote efficient release. These alternatives often compromise taste, flavor, sensorial characteristics of the chewing gum composition, can lead to increase production costs, and ultimately diminish the opportunity for delivering the benefits that a consumer can receive from the chewing gum composition. As a result, formulating for the effective release of active compounds from chewing gum compositions is gathering attention.

Therefore, a need exists for a chewing gum that improves the release characteristics of hydrophilic additives such as salts, vitamins, sweeteners, flavors and other medicaments for consumer and/or health benefits.

### SUMMARY

The present disclosure generally relates to chewing gum compositions. More specifically, the present disclosure relates to chewing gum compositions that improve the release of hydrophilic additives.

In an embodiment, the present disclosure provides a chewing gum comprising at least one wettable filler. The wettable filler aids in producing an increased release of one or more hydrophilic additives.

In an embodiment, the wettable filler can have a γ⁻ greater than 15.0 mJ/m². The wettable filler may be a phyllosilicate. Alternatively, the wettable filler may include, for example, monocalcium phosphate, dicalcium phosphate dihydrate, anyhydrous dicalcium phosphate, tricalcium phosphate, octacalcium phosphate, tetracalcium phosphate, smectite, muscovite or combinations thereof.

In an embodiment, the hydrophilic additive having improved release includes, for example, vitamins, salts, sweeteners, flavors, medicaments or combinations thereof. The hydrophilic additive may include, for example, choline, lipoic acid, inositol, B₁ (Thiamine, Sulbutiamine, Benfotiamine), B₂ (Riboflavin), B₃ (Niacin, Nicotinamide), B₅ (Pantothenic acid, Dexpanthenol, Pantethine), B₆ (Pyridoxine, Pyridoxal phosphate), B₇ (Biotin), B₉ (Folic acid), B₁₂ (Cyanocobalamin, Hydroxocobalamin, Mecobalamin) and combinations thereof.

In an embodiment, the hydrophilic additive may also include, for example, sucralose, aspartame, NAPM derivatives such as neotame, salts of acesulfame, Twinsweet (aspartame-acesulfame salt), altitame, saccharin and its salts, cyclamic acid and its salts, glycyrrhizin, dihydrochalcones, thaumatin, monellin or combinations thereof.

In an embodiment, the hydrophilic additive is coated, encapsulated, agglomerated or absorbed.

In another embodiment, the present disclosure provides a chewing gum comprising a wettable filler having a γ⁻ in a range of at least 25.0 mJ/m² to about 65.0 mJ/m².

In yet another embodiment, the present disclosure provides a chewing gum comprising a wettable filler having a γ⁻ in a range of at least 15.0 mJ/m² to about 65.0 mJ/m². The wettable filler aids in increasing the release at least one hydrophilic additive. The hydrophilic additive may include, for example, vitamins, salts, sweeteners, flavors, medicaments and combinations thereof.

In an embodiment, the wettable filler may include, for example, tricalcium phosphate, octacalcium phosphate, tetracalcium phosphate or combinations thereof.

Alternatively, the wettable filler may include, for example, anhydrous dicalcium phosphate, smectite, muscovite or combinations thereof.

In an embodiment, the hydrophilic additive may include, for example, vitamin C, ascorbic acid and salts thereof, or combinations thereof. The hydrophilic acid may also include, for example, calcium, potassium, sodium ammonium, pyrophosphate, zinc and copper salts or combinations thereof.

In a further embodiment, the present disclosure provides a chewing gum comprising at least one wettable filler having a γ⁻ greater than 15.0 mJ/m². The wettable filler aids in improving the release of one or more hydrophilic additives such as, for example, calcium citrate, potassium phosphate, sodium phosphate or combinations thereof.

In another embodiment, the present disclosure provides a method for increasing the release of hydrophilic additives in a gum composition. The method comprises adding at least one wettable filler to a chewing gum composition comprising one or more hydrophilic additives. The wettable filler aids in increasing the release of the one or more hydrophilic additives. The hydrophilic additive may be, for example, calcium, potassium, sodium, ammonium, pyrophosphate salts or combinations thereof. Alternatively, the hydrophilic additive is calcium citrate. Further, the hydrophilic additive may be, for example, brazzein, luo han guo, steviol glycosides, rebaudioside A, Rebiana, monatin or combinations thereof.

In an embodiment, the rate of release of one or more hydrophilic additives is greater than about six minutes during mastication of the gum composition.

In an embodiment, the hydrophilic additive is a fruit flavor.

An advantage of the present disclosure is to provide an improved chewing gum composition.

Another advantage of the present disclosure is to provide a more optimal release of a variety of hydrophilic materials in chewing gums.

A further advantage of the present disclosure is to provide a chewing gum composition that promotes a more complete and late release of hydrophilic materials contained in the composition.

Still another advantage of the present disclosure is to provide a chewing gum composition with improved sensorial benefits.

Yet another advantage of the present disclosure is to provide a chewing gum composition with improved health benefits.

Another advantage of the present disclosure is to provide a method for increased release of hydrophilic materials contained in a chewing gum composition.

Additional features and advantages are described herein, and will be apparent from the following Detailed Description and the figures.

### BRIEF DESCRIPTION OF THE FIGURES

FIG. 1 lists interfacial and surface tension values for various materials.

FIG. 2 illustrates two plots of time versus calcium and phosphorus levels for comparison of Example 1a against Example 2a.

FIG. 3 illustrates two plots of time versus calcium and phosphorus levels for comparison of Examples 2a and 2b against Example 1a.

FIG. 4 illustrates two plots of time versus calcium and phosphorus levels for comparison of Examples 1c and 1e against Example 1a.

FIG. 5 illustrates two plots of time versus calcium and phosphorus levels for comparison of Examples 1c and 1d against Example 1a.

### DETAILED DESCRIPTION

The present disclosure relates to chewing gum compositions and method of making same.

As used herein, “wetting” is the contact between a fluid and a surface, when the two are brought into contact.

As used herein, “hydrophilic” refers to a physical property of a molecule that can transiently bond with water (H₂O) through hydrogen bonding. The hydrophilic materials and additives disclosed herein are those that possess a hydrophilic molecule or a portion of a molecule that is typically charge-polarized and capable of hydrogen bonding.

As used herein, “uncalibrated chew panel” are subjects that have not undergone calibration to determine statistical repeatability in release using the same gum formula

As used herein, “calibrated chew panel” are subjects that have undergone calibration to determine statistical repeatability in release using the same gum formula

“Late term” release is a time greater than 6 minutes whereby active ingredients continue to release from a chewing gum composition in saliva during mastication.

In accordance with the present disclosure, and as further explained below, it has been found that the use of wettable fillers in chewing gum compositions act in promoting upon mastication of the chewing gum, a more complete and late term release of the hydrophilic material. This effective release subsequently improves sensorial and/or health benefits offered by the chewing gum composition.

It has been found that the cause for the incomplete and retarded release of salts and other compounds such as vitamins, sweeteners, flavors and medicaments, relates to the wettability and non-wettability of the fillers employed in chewing gum compositions.

For example, molecules inside a composition (liquid or solid) are in every direction affected by equal attraction forces, whereas molecules at the surface lack a neighbor at the air phase and therefore have larger attraction forces towards the composition center. This leads to a situation where the interface has excess free energy. This characteristic of excess free energy can take place in any liquid or solid composition. Generally, a system tends to get a minimum of potential energy by minimizing its phase interphase.

Mineral materials such as, for example, calcium carbonate, magnesium silicate and magnesium carbonate serve as fillers for the chewing gum composition for the purpose of reducing cost, texturizing and softening the rubber in the gum base. As a result, fillers are a main constituent of chewing gum compositions. However, fillers generally have not been linked to effective release of hydrophilic substances.

In a chewing gum composition, apart from the fillers, elastomers, polymers, etc. present in the chewing gum matrix, there is a region of interphase separating the many components present in the chewing gum from each other, and this region comprises the area near the interface. The “interface” is the contacting surface where two materials meet and is synonymous with the term “interfacial region”. Thus, achieving the effective release of salts, vitamins, flavors, sweeteners, medicaments, etc. incorporated into the chewing gum composition occurs by understanding the interfacial region interaction in the chewing gum matrix. To achieve this, the filler present in the chewing gum composition must be “wettable” by a liquid, such as saliva in order for hydrophilic materials such as salts to be released from the interfacial region, breaking free from the attraction forces present in the chewing gum matrix.

On the molecular level, surface tension can be interpreted in terms of molecular interactions such as, for example, hydrogen bonding, permanent dipole interactions and London forces. Specifically with regard to hydrogen bonding, hydrogen atoms serve as bridges linking together two atoms of high electron negativity. As a result, hydrogen bonding often occurs in the chewing gum matrix, causing hydrophilic materials and additives, such as salts, to bind in the interfacial region even with exposure to saliva and sheer caused by mastication. Therefore, due to the binding in the interfacial region, the salt release is impeded, along with any sensorial or health benefits it may impart. Accordingly, the present disclosure provides a solution to the binding of hydrophilic materials in chewing gum compositions through the usage of wettable fillers.

A variety of wettable fillers are employable. In an embodiment of the present disclosure, suitable wettable fillers include, for example, phyllosilicates, including serpentines (antigorite, chrysotile, lizardite), clays (kaolinite, illite, smectite, montmorillonite, vermiculite), talc, pyrophyllite, micas (biotite, muscovite, phlogopite, lepidolite, margarite, glauconite) and chlorites.

In another embodiment of the present disclosure, suitable wettable fillers include, for example, monocalcium phosphate, dicalcium phosphate dihydrate (DCPD), dicalcium phosphate anhydrous (DCPA), tricalcium phosphate (TCP), octacalcium phosphate (OCP), tetracalcium phosphate or combination thereof. Of these, dicalcium phosphate serves as the wettable filler in a chewing gum composition for improved and long-term release of salts for oral care benefits.

Alternatively, suitable fillers may be determined based on their wettability characteristics or properties. Quantifying properties used to determine wetting include interfacial tension and surface tension. Interfacial tension refers to the amount of surface free energy existing between two immiscible liquid phases. Surface tension, caused by the attraction between the molecules of a liquid by various intermolecular forces, is a property of the surface of the liquid that causes it to behave as an elastic sheet. The properties of interfacial tension and surface tension assist in determining factor γ⁻, which is an indicator of a filler's wettability. Determining γ⁻ first requires relating the interfacial free energy at a solid-liquid interface (sl) to surface tension at this interface by:

ΔGₛₗ^{IF}=−2γₛₗ Equation1

This surface tension is defined by the sum of the apolar (LW) and polar (Lewis acid-base, AB) components as follows:

γₛₗ=γₛₗ^{LW}+γₛₗ^{AB} Equation2

The apolar parameter can be defined further by the relationship between the individual surface tensions:

γₛₗ^{LW}=(\sqrt{γₛ^{LW}}−\sqrt{γₗ^{LW}})² Equation3

Subsequently, the polar component is related to the following surface tensions, where γ⁺ is the lewis acid component and γ⁻ is the lewis base component:

γₛₗ^{AB}=2(\sqrt{γₛ⁺γₛ⁻}+\sqrt{γₗ⁺γₗ⁻}−\sqrt{γₛ⁺γₗ⁻}−\sqrt{γₗ⁺γₛ⁻}) Equation4

By combining Equations 3 and 4 into Equation 2 the full Young's expression for surface tension at a solid-liquid interface is as follows:

$${γ}_{sl} = {( \sqrt{{γ}_{s}^{LW}} - \sqrt{{γ}_{l}^{LW}} )}^{2} + 2 ( \sqrt{{γ}_{s}^{+} {γ}_{s}^{-}} + \sqrt{{γ}_{l}^{+} {γ}_{l}^{-}} - \sqrt{{γ}_{s}^{+} {γ}_{l}^{-}} - \sqrt{{γ}_{l}^{+} {γ}_{s}^{-}} ) \qquad Equation 5$$

From the Young's expression, the boundary between a hydrophilic or hydrophobic solid is delineated when equivalent apolar and polar surface tension contributions are provide to total interfacial free energy ΔGₛₗ^{IF}:

γₛₗ^{LW}=−γₛₗ^{AB} Equation6

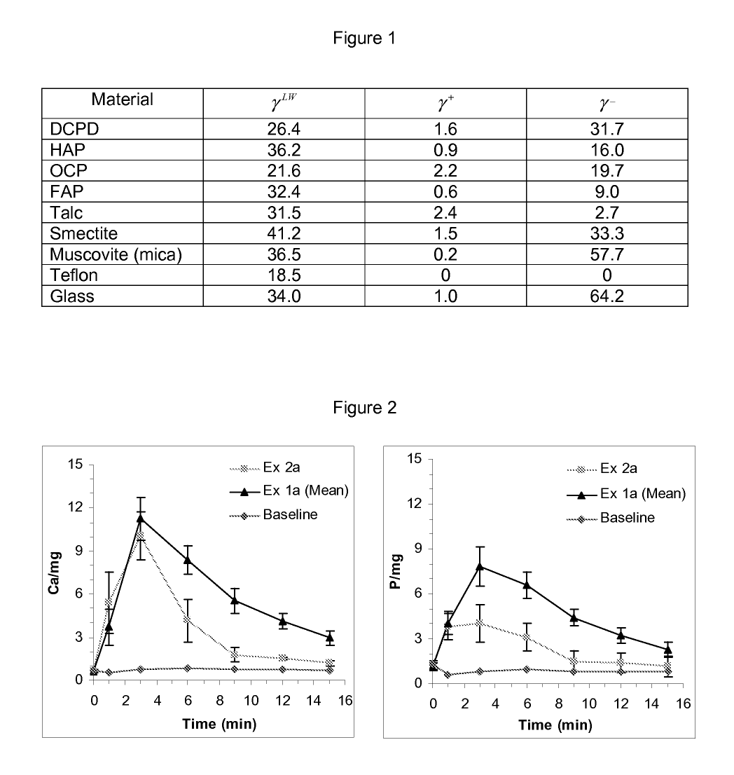

Further, with the restriction in Equation 6 and substituting tension values, γ^{LW} and γ⁺, representative of a typical mineral into FIG. 1, the hydrophilic-hydrophobic boundary can be quantitatively defined in terms of the Lewis base surface tension component γ⁻. For additional detail regarding interfacial tension, refer to van Oss and Giese, “The Hydrophilicity and Hydrophobicity of Clay Minerals”, Clays and Clay Minerals, Volume 3, No. 4, 474-477, 1995 incorporated herein by reference.

Accordingly, in another embodiment of the present disclosure, the wettable fillers preferably have a γ⁻ of at least 15.0 mJ/m², a γ⁻ of at least 25.0 mJ/m², a γ⁻ of at least 35.0 mJ/m², a γ⁻ of at least 45.0 mJ/m², and γ⁻ of at least 55.0 mJ/m². Still further, the wettable fillers of the present disclosure have a γ⁻ of less than 65.0 mJ/m².

In alternative embodiment of the invention, the wettable filler has a γ⁻>28.0 mJ/m². Employing, in a chewing gum composition, a wettable filler having a γ⁻>28.0 mJ/m² provides increased and long term release of calcium, phosphate, pyrophosphate, potassium, copper, ammonium and zinc salts alone or in combination with oral care benefits such as, for example, remineralization, tooth sensitivity benefits, hypersensitivity benefits, anti-caries, plaque removal, plaque neutralization, anti-tartar/calculus agents, halitosis benefits, tooth whitening, anti-inflammatory benefits, gingivitis benefits or combinations thereof.

In various embodiments, one may employ more than one wettable filler in a chewing gum composition for aiding in the release of hydrophilic materials from the chewing gum during mastication, each having a γ⁻ of at least 15.0 mJ/m².

Wettable fillers are also employable in a chewing gum composition for improved release of a sweetener. Sweeteners may include, for example, sucralose, aspartame, NAPM derivatives such as neotame, salts of acesulfame, Twinsweet (aspartame-acesulfame salt), altitame, saccharin and its salts, cyclamic acid and its salts, glycyrrhizin, dihydrochalcones, thaumatin, monellin or combinations thereof. Sweeteners may also include natural sweeteners such as, for example, brazzein, luo han guo, steviol glycosides, rebaudioside A, Rebiana, monatin, or combinations thereof.

In an embodiment, a chewing gum composition comprises a wettable filler having a γ⁻>15.0 mJ/m² to increase the release of sweeteners such as, for example, Twinsweet (aspartame-acesulfame salt), salts of acesulfame, salts of cyclamic acid and salts of saccharin or combinations thereof.

In still another embodiment of the invention, the wettable filler employed in the chewing gum composition increases the release of a hydrophilic additive such as, for example, a water soluble vitamin such as, for example, both natural and artificial sources of vitamin C, ascorbic acid and salts thereof, choline, lipoic acid, inositol, B₁ (Thiamine, Sulbutiamine, Benfotiamine), B₂ (Riboflavin), B₃ (Niacin, Nicotinamide), B₅ (Pantothenic acid, Dexpanthenol, Pantethine), B₆ (Pyridoxine, Pyridoxal phosphate), B₇ (Biotin), B₉ (Folic acid), B₁₂ (Cyanocobalamin, Hydroxocobalamin, Mecobalamin), or combinations thereof. Further, the release of vitamin C, B₆, and B₁₂ are improved by employing a wettable filler having a γ⁻>15.0 mJ/m² in a chewing gum composition.

The chewing gum composition of the present disclosure employs wettable fillers in a range of at least about 0.01% by weight, at least 2.0% by weight, at least 4.0% by weight, at least 8.0% by weight, at least 10.0% by weight or even at least 15.0% by weight.

Typically, chewing gums comprise two phases, a water insoluble portion primarily known as chewing gum base, and a water-soluble portion. The water-soluble portion can include bulk sweeteners, high intensity sweeteners, flavoring agents, softeners, emulsifiers, colors, acidulants, fillers, antioxidants, and other components that provide desired attributes.

The wettable filler may be included in the chewing gum base portion, the water-soluble portion, or both. Generally, the gum base has low or no filler or wettable filler content. When included, the gum base may contain 0.001% to about 1.00% filler and/or 0.001% to about 1.00% wettable filler. When incorporated into the chewing gum composition as a separate component, the wettable filler generally is not in contact with the gum base until mixed to make the final chewing gum composition.

The insoluble gum base generally comprises elastomers, resins, fats and oils, softeners, and inorganic fillers and may include wax. The insoluble gum base may constitute approximately 5% to about 95% by weight of the chewing gum. However, the gum base typically constitutes from about 10% to about 50% of the chewing gum and, more typically, from about 25% to about 35% by weight of the chewing gum.

In various embodiments, the chewing gum base contains from about 20% to about 60% by weight of a synthetic elastomer, up to about 30% by weight of a natural elastomer, from about 5% to about 55% by weight of an elastomer plasticizer, from about 0.01% to about 35% by weight of a filler, from about 5% to about 35% by weight of a softener, and optional minor amounts (e.g., about 1% or less by weight) of miscellaneous ingredients such as colorants, antioxidants, etc.

Synthetic elastomers may include, for example, polyisobutylene having a GPC weight average molecular weight of about 10,000 to about 95,000, isobutylene-isoprene copolymer (butyl elastomer), styrene-butadiene copolymers (having styrene-butadiene ratios of, for example, about 1:3 to about 3:1), polyvinyl acetate having GPC weight average molecular weight of about 2,000 to about 90,000, polyisoprene, polyethylene, vinyl acetate-vinyl laurate copolymer having vinyl laurate content of about 5% to about 50% by weight of the copolymer, and combinations thereof.

Preferred synthetic elastomers include polyisobutylene having a GPC weight average molecular weight of from about 50,000 to 80,000, styrene-butadiene copolymers having a styrene-butadiene ratio for bound styrene of from 1:1 to 1:3, polyvinyl acetate having a GPC weight average molecular weight of from 10,000 to 65,000, with the higher molecular weight polyvinyl acetates typically used in bubble gum base, and vinyl acetate-vinyl laurate copolymer having a vinyl laurate content of 10.

Natural elastomers may include natural rubber, such as smoked or liquid latex and guayule, as well as natural gums, such as jelutong, lechi caspi, perillo, sorva, massaranduba balata, massaranduba chocolate, nispero, rosindinha, chicle, gutta hang kang, and combinations thereof. Synthetic elastomer and natural elastomer concentrations in the base vary depending on whether the chewing gum is adhesive or conventional, bubble gum or regular gum. Preferred natural elastomers include jelutong, chicle, sorva, and massaranduba balata.

Elastomer plasticizers may include, but are not limited to, natural rosin esters such as glycerol esters or partially hydrogenated rosin, glycerol esters of polymerized rosin, glycerol esters of partially dimerized rosin, glycerol esters of rosin, pentaerythritol esters of partially hydrogenated rosin, methyl and partially hydrogenated methyl esters of rosin, pentaerythritol esters of rosin; synthetics such as terpene resins derived from alpha, beta, and/or any suitable combinations of the foregoing. The elastomer plasticizers used will also vary depending on the specific application and type of elastomer used.

Basic fillers and/or texturizers may include, for example, inorganic powders such as magnesium and calcium carbonate, ground limestone, silicate types such as magnesium and aluminum silicate, clay, alumina, talc, titanium oxide, mono-, di- and tri-phosphate, cellulose polymers, such as wood, and combinations thereof. As stated above, at least a portion of the filler of the present disclosure is wettable, having a γ⁻>15.0 mJ/m².

Softeners and/or emulsifiers may include tallow, hydrogenated tallow, hydrogenated and partially hydrogenated vegetable oils, cocoa butter, glycerol monostearate, glycerol triacetate, lecithin, mono and triglycerides, acetylated monoglycerides, fatty acids (e.g. stearic, palmitic, oleic and linoleic acids), and combinations thereof.

Colorants and whiteners may include FD&C dyes and lakes, fruit and vegetable extracts, titanium dioxide, and combinations thereof.

The gum base may include wax. However, U.S. Pat. No. 5,286,500 discloses an example of a wax-free gum base, the disclosure of which is incorporated herein by reference.

Beside the water insoluble gum base portion, a typical chewing gum composition further includes a water-soluble bulk portion. The water-soluble portion can include, for example, bulk sweeteners, high intensity sweeteners, flavoring agents, softeners, emulsifiers, colors, acidulants, fillers, antioxidants, and other components that provide desired attributes.

Softeners typically optimize the chewability and mouthfeel of the chewing gum. The softeners, also known as plasticizers and plasticizing agents, generally constitute from about 0.5% to about 15% by weight of the chewing gum. The softeners may include glycerin, lecithin, and combinations thereof. Aqueous sweetener solutions such as those containing sorbitol, hydrogenated starch hydrolysates (e.g., hydrogenated starch hydrolysate syrups or maltitol syrups), corn syrup, and combinations thereof may also be used as softeners and binding agents in the chewing gum. Aqueous softeners may be combined with glycerin or propylene glycol to produce co-evaporated syrups such as those described, for example, in U.S. Pat. No. 4,671,961.

An emulsifier may be incorporated to improve the consistency and stability of the gum product. An emulsifier can also contribute to product softness. Lecithin is the most commonly employed emulsifier, although nonionic emulsifiers such as polyoxyethylene sorbitan fatty acid esters and partial esters of common fatty acids (lauric, palmitic, stearic and oleic acid hexitol anhydrides (hexitans and hexides) derived from sorbitol may also be used. When used, emulsifiers typically comprised 0.5 to 2% of the chewing gum composition.

The chewing gum compositions of the present disclosure may also include surface active agents. These include, for example, salts of potassium, ammonium, or sodium. Sodium salts include anionic surface active agents, such as alkyl sulfates, including sodium lauryl sulfate, sodium laureth sulfate, and the like. Other sodium salts include sodium lauroyl sarcosinate, sodium brasslate, and the like. Suitable ammonium salts include betaine derivatives such as cocamidopropyl betaine, and the like.

Chewing gums may have added moisture as a separate ingredient, but it is typically a byproduct of the moisture contents of other ingredients. While almost all food ingredients contain some water, carbohydrate syrups contribute most of the water. Other components that may contribute significant amounts of moisture include, for example, certain bulking agents, glycerin and occasionally other ingredients. The total amount of moisture in a chewing gum product affects its texture and stability and, if packaging does not protect sufficiently the product, undesired moisture loss may occur. Initial moisture levels in chewing gums may be as little as 0.1%, by weight, or even less, or as high as 3 to 4%, by weight, depending on the type of gum, the ingredients used, the intended geographic market, the presence of moisture sensitive ingredients and other factors. Pellet centers typically exhibit relatively low moisture levels, while sugar stick gums often exhibit relatively high moisture levels.

Bulk sweeteners, or bulking agents, include both sugar and sugarless components. Bulk sweeteners typically constitute from about 5% to about 95% by weight of the chewing gum, more typically from about 20% to about 80% by weight of the chewing gum and, more typically, from about 30% to about 60% by weight of the gum. Sugar sweeteners generally include saccharide components commonly known in the chewing gum art, including but not limited to, sucrose, dextrose, maltose, dextrin, dried invert sugar, fructose, levulose, galactose, corn syrup solids, and the like, alone or in combination. Sugarless sweeteners include, but are not limited to, sugar alcohols such as sorbitol, mannitol, xylitol, maltitol, hydrogenated starch hydrolysates, erythritol, tagatose, trehalose, and the like, alone or in combination.

High intensity artificial sweeteners can function alone, or in combination, with the above bulk sweeteners. High intensity artificial sweeteners include, for example, sucralose, aspartame, Twinsweet (aspartame and acesulfame salt), NAPM derivatives such as neotame, salts of acesulfame, altitame, saccharin and its salts, cyclamic acid and its salts, glycyrrhizin, dihydrochalcones, thaumatin, monellin, and the like, alone or in combination. Natural sweeteners including but limited to brazzein, luo han guo, steviol glycosides, rebaudioside A, Rebiana, monatin may also be employed.

The chewing gum may incorporate combinations of sugar and/or sugarless sweeteners. Additionally, the softener may also provide additional sweetness such as with aqueous sugar or alditol solutions.

If making a low calorie gum, one can use a low calorie bulking agent such as, for example, polydextrose, raftilose, raftilin, fructooligosaccharides (e.g., NutraFlora®), Palatinose oligosaccharide, guar gum hydrolysate (e.g., Sun Fiber®), or indigestible dextrin (e.g., Fibersol®).

In order to provide longer lasting sweetness and flavor perception, it may be desirable to encapsulate or otherwise control the release of at least a portion of the sweetener employed. Techniques such as wet granulation, wax granulation, spray drying, spray chilling, fluid bed coating, coacervation, and fiber extension can achieve the desired release characteristics.

Optionally, the chewing gum of the present disclosure may include additional breath freshening, anti-microbial or oral health ingredients, such as food acceptable metallic salts selected from zinc and copper salts of gluconic acid, zinc and copper salts of lactic acid, zinc and copper salts of acetic acid, zinc and copper salts of citric acid, copper chlorophyll and combinations thereof. Chewing gums of the present disclosure may also include one or more food acids (e.g., ascorbic acid) that typically provide a tart, or sour, flavor to fruit-flavored products. A particular food acid, and its concentration in the product, may control the nature and release of tartness in the product.

Chewing gum generally conveys oral care benefits. In addition to mechanical cleaning of the teeth provided by the chewing action, saliva stimulated by chewing, flavor and taste from the product conveys beneficial properties in reducing bad breath, neutralizing acid, and remineralizing teeth. Saliva also contains beneficial polypeptides and other components that may improve the oral environment. These include, for example, antimicrobial proteins such as lysozyme, lactoferrin, peroxidases, and histatins as well as inhibitors of spontaneous crystallization, such as statherin.

To assist in providing these benefits, chewing gums of the present disclosure may serve as vehicles for the delivery of specialized oral care agents by using the wettable fillers as described herein.

Oral care agents having improved delivery and extended release through incorporation of wettable fillers may include, for example, antimicrobial compounds such as Cetylpyridinium Chloride (CPC), triclosan, chlorhexidine, and magnolia bark extract (MBE); anti-caries agents such as calcium and phosphate ions, plaque removal agents such as abrasives, surfactants and enzymes; plaque neutralization agents such as ammonium salts, urea and other amines; anti-tartar/calculus agents such as soluble pyrophosphates salts; anti halitosis agents such as parsley oil and copper or zinc salts of gluconic acid, lactic acid, acetic acid or citric acid, and whitening agents such as peroxides; agents that may provide either local or systemic anti-inflammatory effects to limit gingivitis, such as COX-2 inhibitors; agents that may reduce dentinal hypersensitivity, such as potassium salts to inhibit nerve cell transmission, and calcium phosphate salts to block the dentinal tubules.

In a further embodiment of the present disclosure, the hydrophilic additive released may be encapsulated or coated to delay or increase the release rate. Methods for obtaining an encapsulated or coated hydrophilic additive include, for example, (1) encapsulation (either fully or partially), (2) agglomeration, (3) fixation or absorption, and (4) entrapment into a extruded compound. These four methods may operate alone or in combination in any usable manner that physically modifies the release or dissolvability of hydrophilic additive in conjunction with a wettable filler included in this invention.

In an embodiment, hydrophilic additives employed are encapsulated or coated with a barrier layer. Physical modifications of the hydrophilic additives by encapsulation with another substrate may increase or delay their release by modifying the solubility or dissolution rates of the hydrophilic additive. On can use any standard technique that gives full or partial encapsulation. These techniques include, for example, spray drying, spray chilling, fluid-bed coating, extrusion, coextrusion, inclusion, granulation, roll compaction and coacervation. These encapsulation techniques, which give full or partial encapsulation, can operate individually or in combination in a single step process or multiple step process.

Coating or encapsulating the hydrophilic additives herein described generally requires standard coating techniques with varying degrees of coating, from partial to full coating, depending on the coating composition used in the process. In addition, the compositions may be susceptible to water permeation of varying degrees. Generally, composition having high organic solubility, good film forming properties and low water solubility give a better delayed release. Such compositions include, for example, acrylic polymers and copolymers, carboxyvinyl polymer, polyamides, polysterene, polyvinyl acetate, polyvinyl acetate phthalate, polyvinylpyrrolidone and waxes. Although all of these materials can serve as encapsulants, typically only food-grade materials should be considered.

Agglomeration is another method for modifying the release of hydrophilic additives. Agglomeration requires an agglomerating agent to coat partially the hydrophilic additives. This method also includes mixing an additive and an agglomerating agent with a small amount of water or solvent. The mixture is prepared in such a way as to have individual wet particles in contact with each other to apply a partial coating. After removing the water or solvent, the mixture is ground and used as a powdered, coated product.

Agglomerating agents are the same as those used in encapsulation procedures mentioned previously. However, since the coating is only a partial encapsulation, some agglomerating agents are more effective in modifying the release of hydrophilic additives than others. Suitable agglomerating agents include, for example, organic polymers like acrylic polymers and copolymers, polyvinyl acetate (PVAc), polyvinylpyrolidone, waxes, shellac, and zein. Other agglomerating agents include, for example, agar, alginates, a wide range of cellulose derivatives like ethyl cellulose, methyl cellulose, sodium hydroxymethylcellulose, hydroxypropylmethyl cellulose, dextrin, gelatin, modified and unmodified starches, and vegetable gums like guar gum, locust bean gum, and carrageenan. The level of agglomerating agent may be, for example, at least 5% by weight of agglomeration matrix.

In another embodiment, the hydrophilic additive may be absorbed onto another component that is porous and becomes entrapped in the matrix of the porous component. Common materials used for absorbing the hydrophilic material include, for example, silicas, silicates, pharmasorb clay, sponge-like beads or microbeads, amorphous sugars like spray-dried dextrose, sucrose, alditols, amorphous carbonates and hydroxides including aluminum and calcium lakes, vegetable gums and other spray dried materials.

Depending on the type and preparation of absorbent material, the amount of hydrophilic material loadable onto the absorbent will vary. Generally, materials like polymers or sponge-like beads or microbeads, amorphous sugars and alditols and amorphous carbonates and hydroxides absorb an amount equal to about 10% to about 40% of the weight of the absorbent. Other materials like silicas and pharmasorb clays may be able to absorb about 20% to about 80% of the weight of the absorbent.

After a hydrophilic additive is absorbed onto an absorbent or fixed onto an absorbent, the additive can be coated by encapsulation, either or fully or partially, as described above.

Alternatively, entrapment of an ingredient by fiber extrusion or fiber spinning into a polymer is another form of encapsulation.

### EXAMPLES

By way of example and not limitation, the following examples are illustrative of various embodiments of the present disclosure. Specifically, the following examples compare release profiles in various chewing gum formulas to demonstrate a trend of incremental increase in release inhibition of hydrophilic additives such as salts relative to use, level or use and type of various fillers.

A. Background

To meet consumer acceptance criterion, four different pilot plant chewing gum batches containing the same type and level of remineralizing agents were produced. Based on analytical results from post-consumer tests, each of the four pilot plant batches, all containing the same type and level of remineralizing agents, were found to not match the original remineralization chewing gum formula profile used, and proved efficacious during previous clinical testing. Upon further review, the modified consumer test formulas utilized different flavors, bases, mixing procedures, and removed extraneous dicalcium phosphate (anhydrous) filler. Therefore, testers conducted four experimental series to determine which factors were influencing release of the remineralization agents.

B. Technical Hypothesis

In consumer testing, the remineralization agents, calcium and phosphate did not show the same level of late term release of the original remineralization formula used during clinical testing. Since migration and retention governs the release of many ingredients, such as salts, vitamins and medicaments, testers conducted systematic substitutions of flavor, base, and filler type and mixing procedures with the original remineralization formula to determine which factors were influencing gum softness and wettability.

C. Materials & Resources Used

TABLE 1

| Ingredient | Ex. 1a | Ex. 1b | Ex. 1c | Ex. 1d | Ex. 1e | Ex. 2a | Ex. 2b |
|---|---|---|---|---|---|---|---|
| Sorbitol | 39.00 | 39.00 | 39.00 | 39.00 | 39.00 | 44.00 | 40.00 |
| Base B | 30.00 | 30.00 | 30.00 | 30.00 | 30.00 | 29.00 | 29.00 |
| Calcium citrate | 7.50 | 7.50 | 7.50 | 7.50 | 7.50 | 7.50 | 7.50 |
| Encapsulated Sodium | 7.50 | 7.50 | 7.50 | 7.50 | 7.50 | 7.50 | 7.50 |
| phosphate diabasic | | | | | | | |
| (47%)/Potassium | | | | | | | |
| phosphate monobasic | | | | | | | |
| (13%) | | | | | | | |
| Glycerin | 5.50 | 5.50 | 5.50 | 5.50 | 5.50 | 3.00 | 3.00 |
| Dicalcium Phosphate | 4.00 | * | * | * | * | * | 4.00 |
| Talc | * | * | 4.00 | * | * | * | * |
| Calcium Carbonate | * | * | * | * | 4.00 | * | * |
| Tricalcium Phosphate | * | * | * | 4.00 | * | * | * |
| Xylitol | * | * | * | * | * | 4.80 | 4.80 |
| Flavor | 1.74 | 1.74 | 1.74 | 1.74 | 1.74 | 1.70 | 1.70 |
| Color | * | * | * | * | * | 1.00 | 1.00 |
| Menthol | 0.54 | 0.54 | 0.54 | 0.54 | 0.54 | 0.25 | 0.25 |
| Triacetin | 0.25 | 0.25 | 0.25 | 0.25 | 0.25 | 0.25 | 0.25 |
| Lecithin | 0.40 | 0.40 | 0.40 | 0.40 | 0.40 | 0.40 | 0.40 |
| High intensity | 3.27 | 3.27 | Ex. 1a | Ex. 1a | Ex. 1a | 0.50 | 0.50 |
| sweeteners | | | | | | | |

TABLE 2

| Ingredient | Ex. 3a | Ex. 3b | Ex. 3c | Ex. 3d |
|---|---|---|---|---|
| Sorbitol | 39.00 | 39.00 | 39.00 | 39.00 |
| Base A | 30.00 | * | 30.00 | * |
| Base B | * | 30.00 | * | 30.00 |
| Calcium citrate | 7.50 | 7.50 | 7.50 | 7.50 |
| Encapsulated Sodium | 7.50 | 7.50 | 7.50 | 7.50 |
| phosphate diabasic | | | | |
| (47%)/Potassium phos- | | | | |
| phate monobasic (13%) | | | | |
| Glycerin | 5.50 | 5.50 | 5.50 | 5.50 |
| Flavor A | 1.74 | 1.74 | * | * |
| Flavor B | * | * | 1.74 | 1.74 |
| Menthol | 0.54 | 0.54 | 0.54 | 0.54 |
| Triacetin | 0.25 | 0.25 | 0.25 | 0.25 |
| Lecithin | 0.40 | 0.40 | 0.40 | 0.40 |
| Cooling Agents | 0.35 | 0.35 | 0.35 | 0.35 |
| High intensity | 3.27 | 3.27 | 3.27 | 3.27 |
| sweeteners | | | | |

D. Test Methods and Results

1. Measurement of Release Kinetics

Seven collection tubes yielding 50 ml of saliva per subject were weighed and labeled consecutively with T=0, 1, 3, 6, 9, 12 and 15 minutes. For the T=0 tube, unstimulated saliva (saliva generated by a subject without the mastication of chewing gum, base, or parafilm to enhance saliva flow) collected for 10 minutes determined baseline calcium (Ca) and phosphorous (P) levels. Two gum pieces were then weighed with gum stimulated saliva collected during the following time intervals in minutes: 0-1, 1-3, 3-6, 6-9, 9-12 and 12-15. Tubes with collected saliva were then re-weighed, saliva mass was calibrated and elemental analysis measured externally by ICP (Coupled Plasma Spectroscopy) to determine Ca and P concentrations (ppm).

Calcium and phosphorous salivary concentrations were converted from part per million (ppm) to milligrams (mg) using saliva mass. The conversion eliminates the noise in the concentration data due to saliva flow variability among subjects.

Using formula Example 1a in Table 1, Ca and P release profiles for 17 trials were generated using a calibrated chew panel. As used herein, a “calibrated chew panel” includes subjects that have undergone calibration to determine statistical repeatability in release using the same gum formula.

TABLE 3
Statistical analysis for release of Ca & P from formula 1a

| Time (min) | Ca (mg) | Std. Dev | p-value | CI |
|---|---|---|---|---|
| 0 | 0.63 | 0.11 | 0.99 | 0.05 |
| 1 | 3.72 | 1.25 | | 0.59 |
| 3 | 11.23 | 1.46 | | 0.70 |
| 6 | 8.36 | 0.99 | | 0.47 |
| 9 | 5.55 | 0.87 | | 0.41 |
| 12 | 4.11 | 0.56 | | 0.26 |
| 15 | 2.94 | 0.51 | | 0.24 |
| TOTAL | 36.55 | | | |
| Time (min) | P (mg) | Std. Dev | p-value | CI |
| 0 | 1.12 | 0.25 | 0.99 | 0.12 |
| 1 | 4.05 | 0.78 | | 0.37 |
| 3 | 7.80 | 1.31 | | 0.62 |
| 6 | 6.56 | 0.89 | | 0.42 |
| 9 | 4.40 | 0.56 | | 0.27 |
| 12 | 3.25 | 0.51 | | 0.24 |
| 15 | 2.29 | 0.50 | | 0.24 |
| TOTAL | 29.48 | | | |

The statistical analysis in Table 3 indicates (1) formula Ex. 1a provided consistent batch to batch delivery of the Ca and P ions to saliva (p=0.99) at 95% confidence using an ANOVA model, (2) the 17 trials provided a benchmark release profile mean with standard deviation and (3) the release kinetics profile offers a quantitative means for determining whether release of Ca and P is impacted by gum formula modifications.

2. Flavor-Base Design of Experiment (DOE)—Calibrated Panel

Formula Ex. 2a in Table 1 showed a reduced late term release of Ca and P into saliva in comparison to Ex. 1a previously used in clinical testing. FIG. 2 illustrates the comparison between the Ex. 1a and 2a. Upon review of the formulas, different types or sources of flavor and gum base were utilized. Systematic replacement of flavor and base ingredients served to determine their impact on release of the calcium and phosphate salts from the chewing gum compositions. As a result, a 2×2 factoral design assessed whether these modifications may have an impact on release of the Ca and P from the chewing gum compositions into saliva.

Table 2 shows the test matrix where calcium and phosphate profile release trends were generated for Examples 3a, 3b, 3c and 3d using the same methodology for measuring release kinetics as tested in Ex. 1a of Table 1. The profile trend indicated that an individual substitution or a combination of alternate Base A or Flavor A using Base B and/or Flavor B did not influence release.

3. Filler-Mixing DOE—Calibrated Panel

Formula Ex. 2a showed reduced late term release of calcium and phosphate into saliva compared to formula Ex. 1a. Using formula Examples 1a, 1b, 2a and 2b, chewing gum compositions were compared based on the formulas containing or removing dicalcium phosphate as a filler. Moreover, in view of the inclusion or removal of dicalcium phosphate, formulas 1a, 1b, 2a and 2b were compared based on their respective mixing procedures as indicated in Tables 4 and 5 below.

TABLE 4
1a and 1b mix procedures

| Time (minutes) | Step |
|---|---|
| 0-2 | Add base and sorbitol |
| 2 | Add dicalcium phosphate, glycerin, lecithin, |
| | and triacetin |
| 3 | Add flavor and cooling compounds |
| 8 | Add calcium citrate, sodium and potassium |
| | phosphate blend, and high intensity sweeteners |
| 8-13 | Continue mixing; stop mixing after 13 minutes |

TABLE 5
2a and 2b mix procedures

| Time (minutes) | Step |
|---|---|
| 0-2 | Add base and sorbitol |
| 2 | Add dicalcium phosphate, glycerin, lecithin, |
| | and triacetin |
| 3 | Add calcium citrate, sodium and potassium |
| | phosphate blend, and high intensity sweeteners |
| 8 | Add flavor and cooling compounds |
| 8-13 | Continue mixing; stop mixing after 13 minutes |

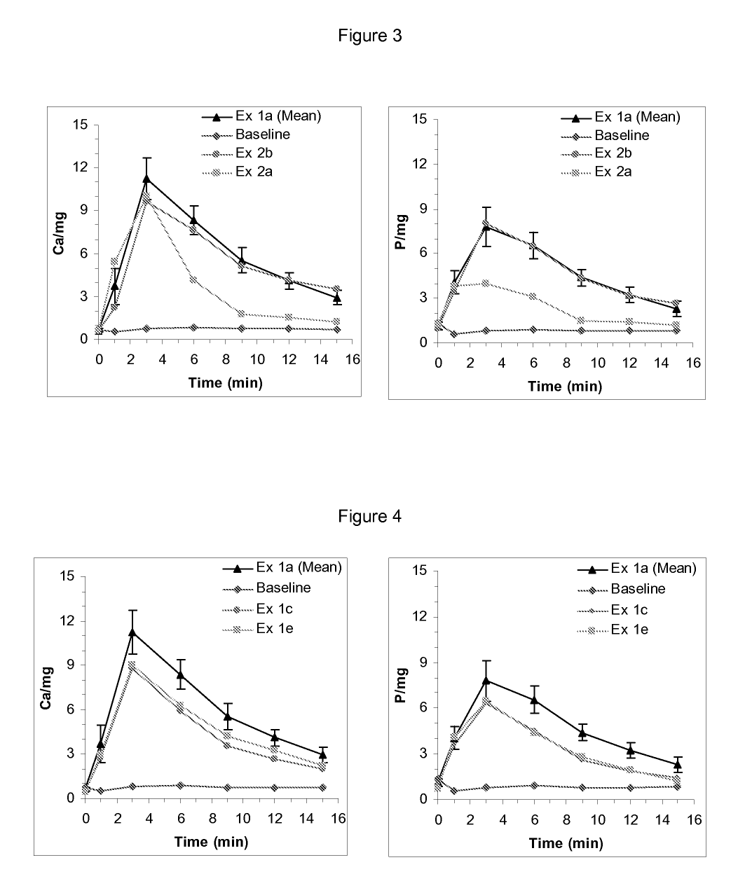

As illustrated in FIG. 3, results indicate that dicalcium phosphate improves delivery of Ca and P. Specifically, the removal of dicalcium phosphate causes a significant reduction in late term delivery of Ca and P irrelevant of the mixing procedures. Moreover, the release profile trend of Ex. 2b (containing dicalcium phosphate) indicates a significant improvement in late term release of Ca and P, whereas the removal of dicalcium phosphate in Ex. 2a reduces late term release of Ca and P.

Further, Table 6 below establishes that the positive release benefits of dicalcium phosphate are due its function properties as a wettable filler and that any increased deliver of Ca and P do not come from the dicalcium phosphate itself. As described in Table 6, the release profile of Ex. 1a provided no appreciable increase in salivary Ca and P levels when the formula removed encapsulated phosphate salts and calcium citrate. This indicates that dicalcium phosphate acts only as a filler and does not release its own calcium and phosphate. Therefore, the use of dicalcium phosphate as a wettable filler in chewing gums is a critical ingredient in providing improved release of soluble calcium and phosphate salts beyond 6 minutes of chewing.

TABLE 6
Comparison of baseline release profiles of Ex. 1a without
calcium citrate and encapsulated phosphate salts, and
with and without anhydrous dicalcium phosphate

| | | | | | |
|---|---|---|---|---|---|
| | Ex. 1a(A) | Ex. 1a(B) | Ex. 1a(A) | Ex. 1a(B) | |
| | With | With | Without | Without | |
| | Anhydrous | Anhydrous | Anhydrous | Anhydrous | |
| Time | dicalcium | dicalcium | dicalcium | dicalcium | |
| (min) | phosphate | phosphate | phosphate | phosphate | Mean |

Ca (mg)

| | | | | | |
|---|---|---|---|---|---|
| 0 | 0.53 | 0.81 | 0.65 | 0.85 | 0.71 |
| 1 | 0.53 | 0.53 | 0.53 | 0.59 | 0.55 |
| 3 | 0.75 | 0.87 | 0.66 | 0.91 | 0.8 |
| 6 | 0.83 | 1.03 | 0.64 | 0.95 | 0.86 |
| 9 | 0.59 | 1.02 | 0.55 | 0.82 | 0.75 |
| 12 | 0.71 | 0.92 | 0.53 | 0.75 | 0.73 |
| 15 | 0.71 | 0.94 | 0.45 | 0.74 | 0.71 |
| Total | 4.65 | 6.13 | 4.02 | 5.62 | 5.1 |

P (mg)

| | | | | | |
|---|---|---|---|---|---|
| 0 | 0.87 | 1.85 | 1.23 | 1.43 | 1.34 |
| 1 | 0.52 | 0.61 | 0.52 | 0.58 | 0.55 |
| 3 | 0.69 | 1.01 | 0.63 | 0.96 | 0.82 |
| 6 | 0.8 | 1.18 | 0.69 | 0.98 | 0.92 |
| 9 | 0.56 | 1.12 | 0.66 | 0.84 | 0.8 |
| 12 | 0.78 | 1.07 | 0.61 | 0.81 | 0.82 |
| 15 | 0.83 | 1.09 | 0.6 | 0.79 | 0.83 |
| Total | 5.05 | 7.93 | 4.94 | 6.38 | 6.08 |

4. Filler Experimentation

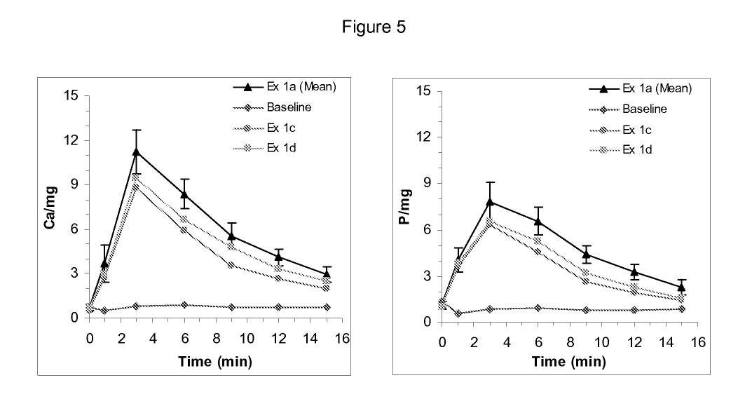

Various fillers were employed to ascertain whether the interfacial theory of wettable fillers could predict the release of soluble materials (such as salts, vitamins and medicaments) from chewing gum compositions. As such, testing a series of characterized gum fillers allowed for analyzing release trends and measuring active release of salts (calcium and phosphorous) using the calibrated panel. FIG. 4 details the resulting trends in release retardation with hydrophobic talc and calcium carbonate fillers in comparison to hydrophilic dicalcium phosphate. Moreover, FIG. 5 details the resulting trends in release improvement when hydrophobicity of fillers such as Tricalcium Phosphate is between highly hydrophobic Talc and hydrophilic Dicalcium Phosphate.

Overall, the release profile supports the theory that that poorly wettable fillers such as talc significantly retards the late term release of salts such as Ca and P from chewing gum and their delivery into saliva during mastication.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1-25. (canceled)

26. A chewing gum comprising at least one wettable filler wherein the wettable filler aids in producing an increased release of one or more hydrophilic additives.

27. The chewing gum of claim 1, wherein the wettable filler has a γ⁻ greater than 15.0 mJ/m².

28. The chewing gum of claim 1, wherein the wettable filler has a γ⁻ less than 65.0 mJ/m².

29. The chewing gum of claim 1, wherein the wettable filler is a phyllosilicate.

30. The chewing gum of claim 1, wherein the wettable filler is selected from the group consisting of monocalcium phosphate, dicalcium phosphate dihydrate, anhydrous dicalcium phosphate, tricalcium phosphate, octacalcium phosphate, tetracalcium phosphate, and combinations thereof.

31. The chewing gum of claim 1, wherein the wettable filler is selected from the group consisting of serpentine, talc, pyrophyllite, mica, chlorite, and combinations thereof.

32. The chewing gum of claim 1, wherein the hydrophilic additive is selected from the group consisting of vitamins, salts, sweeteners, flavors, medicaments, and combinations thereof.

33. The chewing gum of claim 7, wherein the hydrophilic additive is selected from the group consisting of vitamin C, ascorbic acid and salts thereof, choline, lipoic acid, inositol, B₁ (Thiamine, Sulbutiamine, Benfotiamine), B₂ (Riboflavin), B₃ (Niacin, Nicotinamide), B₅ (Pantothenic acid, Dexpanthenol, Pantethine), B₆ (Pyridoxine, Pyridoxal phosphate), B₇ (Biotin), B₉ (Folic acid), B₁₂ (Cyanocobalamin, Hydroxocobalamin, Mecobalamin) and combinations thereof.

34. The chewing gum of claim 7, wherein the hydrophilic additive is selected from the group consisting of sucralose, aspartame, NAPM derivatives, salts of acesulfame, aspartame-acesulfame salt, altitame, saccharin and its salts, cyclamic acid and its salts, glycyrrhizin, dihydrochalcones, thaumatin, monellin and combinations thereof.

35. The chewing gum of claim 1, wherein the hydrophilic additive is coated, encapsulated, agglomerated, or absorbed.

36. A chewing gum comprising a wettable filler having a γ⁻ in a range of at least 15.0 mJ/m² to about 65.0 mJ/m², wherein the wettable filler aids in increasing the release of at least one hydrophilic additive.

37. The chewing gum of claim 11, wherein the wettable filler is selected from the group consisting of octacalcium phosphate, tetracalcium phosphate and combinations thereof.

38. The chewing gum of claim 11, wherein the wettable filler is selected from the group consisting of muscovite, smectite and combinations thereof.

39. The chewing gum of claim 11, wherein the hydrophilic additive is selected from the group consisting of vitamin C, ascorbic acid and salts thereof and combinations thereof.

40. The chewing gum of claim 11, wherein the hydrophilic additive is selected from the group consisting of calcium, potassium, sodium, ammonium, pyrophosphate, zinc and copper salts and combinations thereof.

41. A method of increasing the release of hydrophilic additives in a gum composition comprising: adding at least one wettable filler to a chewing gum composition comprising one or more hydrophilic additives, wherein the wettable filler aids in increasing the release of one or more hydrophilic additives.

42. The method of claim 16, wherein the wettable filler has a γ⁻ in a range of at least 15.0 mJ/m² to about 65.0 mJ/m².

43. The method of claim 16, wherein the wettable filler is selected from the group consisting of dicalcium phosphate dihydrate, anhydrous dicalcium phosphate, octacalcium phosphate, tetracalcium phosphate and combinations thereof.

44. The method of claim 16, wherein the wettable filler is a phyllosilicate.

45. The method of claims 16, wherein the hydrophilic additive is calcium citrate.

46. The method of claim 16, wherein the hydrophilic additive is selected from the group consisting of brazzein, luo han guo, steviol glycosides, rebaudioside A, Rebiana, monatin and combinations thereof.

47. The method of claim 16, wherein the hydrophilic additive is a fruit flavor.

48. The method of claim 16, wherein a rate of release of the one or more hydrophilic additives is greater than about 6 minutes during mastication of the gum composition.
